(19) United States
(12) Patent Application Publication
(10) Pub. No.: US 2022/0040150 A1
(43) Pub. Date: Feb. 10, 2022

(54) INTRAVENOUS DMT ADMINISTRATION METHOD FOR DMT-ASSISTED PSYCHOTHERAPY

(71) Applicant: Universitatsspital Basel, Basel (CH)

(72) Inventor: Matthias Emanuel LIECHTI, Oberwil (CH)

(21) Appl. No.: 17/391,069

(22) Filed: Aug. 2, 2021

Related U.S. Application Data
(60) Provisional application No. 63/061,205, filed on Aug. 5, 2020.

(51) Int. Cl. A61K 31/4045; A61K 9/00; A61P 25/22

(52) U.S. Cl. CPC A61K 31/4045; A61P 25/22; A61K 9/0019

(57) ABSTRACT

A method of inducing a psychedelic state in an individual by administering DMT, a salt of DMT, analogs thereof, or derivatives thereof to an individual with a continuous perfusion system and inducing a psychedelic state. A method of inducing a psychedelic state in an individual safely, by administering DMT, a salt of DMT, analogs thereof, or derivatives thereof to an individual with a continuous perfusion system, inducing a psychedelic state, and adjusting or ending the psychedelic state on demand. A method of providing a short lasting psychedelic treatment of minutes to 1-2 hours, by administering DMT, a salt of DMT, analogs thereof, or derivatives thereof to an individual with a continuous perfusion system and providing psychedelic treatment for minutes to 1-2 hours. A method of determining a dose of DMT for an individual.

### BACKGROUND OF THE INVENTION

### 1. Technical Field

The present invention relates to compositions and methods for administering N,N-dimethyltryptamine (DMT) to induce a psychedelic state and providing therapeutic effects of DMT.

### 2. Background Art

Hallucinogens or psychedelics are substances capable of inducing exceptional subjective effects such as a dream-like alteration of consciousness, affective changes, enhanced introspective abilities, visual imagery, pseudo-hallucinations, synesthesia, altered temporal and special perception, mystical-type experiences, disembodiment and ego dissolution (Holze et al., 2021; Liechti, 2017; Passie et al., 2008).

Psychedelics have also newly been termed psychoplastogens because these substances also exhibit neuroregenerative effects that may contribute to their therapeutic effects (Ly et al., 2018). Neuroplastogenic effects may be present to various extents in a given psychedelic or derivative thereof (Dong et al., 2021).

Psychedelics can be used to assist psychotherapy for many indications including anxiety, depression, addiction, personality disorder and others and can also be used to treat other disorders such as cluster headache and migraine and others (Bogenschutz et al., 2015; Davis et al., 2021; Garcia-Romeu et al., 2015; Gasser et al., 2014; Gasser et al., 2015; Griffiths et al., 2016; Johnson et al., 2014; Krebs & Johansen, 2012; Ross et al., 2016).

There is also evidence that the psychedelic brew Ayahuasca, which contains the active psychedelic substance DMT (Dominguez-Clave et al., 2016) may alleviate depression (de Araujo, 2016; Dos Santos et al., 2016c; Palhano-Fontes et al., 2019; Sanches et al., 2016).

The effects of many psychedelics after oral administration are long-lasting and difficult to control once the substance has entered the body (Holze et al., 2021). This can be problematic as therapeutic sessions may last very long and effects of a psychedelic may be too strong during the process. Thus, a shorter-acting psychedelic and a method of use resulting in a state the intensity of which could be altered or stopped if desired would be highly desirable and could provide a solution for situations in which little time is available and/or a more controlled psychedelic state is to be induced.

DMT (FIG. 1) is a naturally-occurring psychedelic substance widely used in recreational and spiritual settings in the form of Ayahuasca (Dominguez-Clave et al., 2016), a brew that is consumed orally. Similar to LSD or psilocybin, DMT is considered a tool to induce an altered state of consciousness of interest in psychological and psychiatric research (Gallimore & Strassman, 2016; Timmermann et al., 2018). DMT is rapidly metabolized by monoamine oxidase (MAO) A (Riba et al., 2015). Therefore, it is inactive when administered orally and has a very short duration of action when administered parenterally (less than 20 min) (Gallimore & Strassman, 2016; Strassman, 1996; Strassman & Qualls, 1994; Strassman et al., 1994).

In Ayahuasca, DMT is consumed together with harmala alkaloids that inhibit MAO to increase the oral bioavailability of DMT and to prolong its action after oral consumption (Riba et al., 2015). Alternatively, DMT can be administered intravenously as a bolus resulting in a very short action. An intravenous administration regime including a bolus and 1 hour maintenance perfusion has previously been proposed to induce a stable and prolonged DMT experience allowing to study the psychological and autonomic acute effects of DMT (Gallimore & Strassman, 2016). This idea has never been put into practice or further developed into an application or tested in humans and its use has not been specified.

There remains a need for an administration regime to effectively deliver DMT to an individual.

### SUMMARY OF THE INVENTION

The present invention provides for a method of inducing a psychedelic state in an individual by administering DMT, a salt of DMT, analogs thereof, or derivatives thereof to an individual with a continuous perfusion system and inducing a psychedelic state.

The present invention provides for a method of inducing a psychedelic state in an individual safely, by administering DMT, a salt of DMT, analogs thereof, or derivatives thereof to an individual with a continuous perfusion system, inducing a psychedelic state, and adjusting or ending the psychedelic state on demand.

The present invention also provides for a method of providing a short lasting controlled psychedelic treatment of minutes to 1-2 hours, by administering DMT, a salt of DMT, analogs thereof, or derivatives thereof to an individual with a continuous perfusion system and providing psychedelic treatment for minutes to 1-2 hours.

The present invention provides for a method of determining a dose of DMT for an individual, by administering different rates of perfusion of DMT, a salt of DMT, analogs thereof, or derivatives thereof to an individual, and adjusting the dose to provide more positive acute effects than negative acute effects in the individual.

The present invention provides for a method of therapy, by administering an intermediate “good effect dose” of DMT, a salt of DMT, analogs thereof, or derivatives thereof to an individual, and inducing positive acute drug effects that are known to be associated with more positive long-term responses in psychiatric patients.

The present invention further provides for a method of therapy, by administering an “ego-dissolution” dose of DMT, a salt of DMT, analogs thereof, or derivatives thereof to an individual, and providing ego-dissolution.

The present invention provides for a method of adjusting a psychedelic state in real time while effects of DMT have already started in an individual, by adjusting a rate of the DMT perfusion to increase or decrease the intensity and/or duration of the psychedelic state based on the individual's feedback or a therapist's assessment of the individual's state.

### DESCRIPTION OF THE DRAWINGS

Other advantages of the present invention are readily appreciated as the same becomes better understood by reference to the following detailed description when considered in connection with the accompanying drawings wherein:

FIG. 1 is a graph of the chemical structure of DMT;

FIG. 2 shows a table of the different doses schedules of DMT tested in Example 1;

FIG. 3 is a graph of the DMT plasma concentration-time curves targeted with the DMT dosing regimens used in Example 1;

FIGS. 4A-4B show subjective effects of DMT on the VAS scale, FIG. 4A is a graph of any drug effect versus time and FIG. 4B is a graph of good drug effect versus time;

FIGS. 5A-5B show alterations of mind induced DMT and measured with the 5D-ASC scale, FIG. 5A is a graph of bad drug effect versus time and FIG. 5B is a graph of anxiety versus time;

FIG. 6 shows autonomic and adverse effects of DMT; and

FIG. 7A is a graph of systolic blood pressure versus time, FIG. 7B is a graph of diastolic blood pressure versus time, and FIG. 7C is a graph of heart rate versus time.

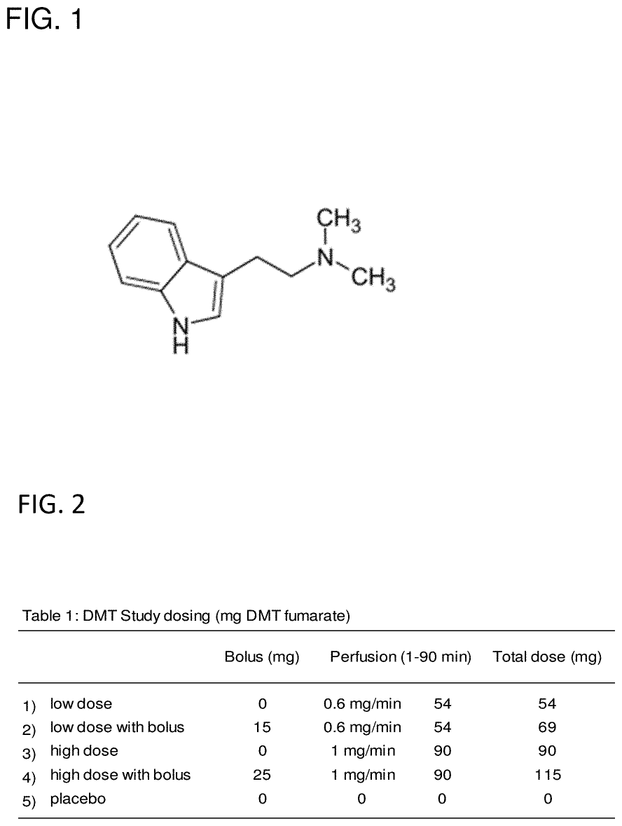

### DETAILED DESCRIPTION OF THE INVENTION

The present invention provides for a method of administering DMT intravenously using continuous administration (perfusion) that can be adjusted over time and even stopped any time if needed. The present invention includes the description of specific dosing schedules derived from previous studies and pharmacokinetic principles, a practical test of the theoretical model in healthy humans (Example 1), and specific uses of such an invention in medical practice for treating psychiatric disorders alone or in combination with psychotherapy.

The use of the intravenous route avoids the complete metabolism of DMT by the liver (first pass effect) when DMT is administered alone without a MAO inhibitor. The use of the continuous intravenous perfusion allows extension of the state compared with a single intravenous bolus DMT administration which would produce effects that last only for a few minutes due to redistribution in the circulation and rapid metabolism of DMT. Additionally, the perfusion method allows for a highly controlled administration compared to an oral administration with a MAO inhibitor allowing also to control the desired effects over side effects similar to the use of intravenous hypnotics (propofol) or analgesics (remifentanil) perfusions during a surgical intervention using total intravenous anesthesia. Optionally, a bolus “loading” dose can be administered before the perfusion to more rapidly reach the desired state compared with a perfusion only administration method. Both approaches have been tested practically in humans in Example 1 within the present invention.

DMT is preferably used in the present invention, however, derivatives and analogs thereof can also be used. Any salt form of DMT can be used. DMT can be found in plants and animals such as Mimosa tenuiflora, Diplopterys cabrerana, and Psychotria viridis, as well as various barks, pods, and beans. DMT in the present invention can be derived from a natural source or synthetically produced. Dosing of DMT and the dosing rationale are further described below.

Pharmacologically, DMT interacts with the serotonin 5-HT_{2A} receptor similar to other classic hallucinogens including LSD and psilocybin (Rickli et al., 2016). Activation of the 5-HT_{2A} receptor is the primary action of psychedelics leading to perceptual alterations in humans (Kraehenmann et al., 2017; Preller et al., 2016; Vollenweider et al., 1998). In contrast to LSD, DMT also interacts with the serotonin transporter (Cozzi et al., 2009; Rickli et al., 2016) and in contrast to psilocybin it exhibits greater affinity to the 5-HT₁ receptor (Rickli et al., 2016). Thus, there are similarities but also some pharmacologically distinct properties of DMT compared to other serotonergic hallucinogens. The main difference of DMT in comparison with LSD or psilocybin is inactivity when administered orally without MAO inhibition and its short action when given intravenously or by inhalation.

The most prevalent form of use of DMT is in the form of orally administered Ayahuasca. As such it is used worldwide and legally in some countries. There is also considerable research on Ayahuasca including therapeutic trials showing efficacy as an antidepressant (de Araujo, 2016; Dos Santos et al., 2016a; Dos Santos et al., 2016b; Palhano-Fontes et al., 2019; Sanches et al., 2016).

Pure DMT is also used recreationally, typically by inhalation (vaping/smoking) (Winstock et al., 2014). In addition, DMT has been administered intravenously within research projects. This form of administration allows the fast and reproducible induction of a transient psychedelic state and provides insights into the structure of the human mind (Gallimore & Strassman, 2016; Gouzoulis-Mayfrank et al., 2005; Strassman & Qualls, 1994; Strassman et al., 1996; Strassman et al., 1994; Timmermann et al., 2019; Timmermann et al., 2018) and is also the focus of the present invention.

Studies of intravenous DMT administration in humans began in the 1950s with a focus on mimicking psychotic states (Boszormenyi & Szara, 1958; Faillace et al., 1967; Szara, 1957; Szara, 2007; Szara et al., 1966). Then, DMT was further investigated in the 1990ies in healthy subjects when research on LSD was prohibited (Gouzoulis-Mayfrank et al., 2005; Heekeren et al., 2007; Strassman & Qualls, 1994; Strassman et al., 1996; Strassman et al., 1994). Currently, only one research group investigates the acute effects of pure intravenous DMT in healthy subjects (Alamia et al., 2020; Schartner & Timmermann, 2020; Timmermann et al., 2019; Timmermann et al., 2018). There is increased interest in the effects of this substance due to the rapid on- and offset of action compared to other substances used as psychedelic research tools and a target-controlled intravenous infusion dosing regime has been proposed which also provides one basis for the present invention (Gallimore & Strassman, 2016).

Previous research already documented the rapid action of DMT and its safety (Gouzoulis-Mayfrank et al., 2005). A study included 15 healthy volunteers. DMT was given at either a low or a high dose. The low dose consisted of a bolus injection of 0.2 mg/kg (˜15 mg) over 5 minutes followed by a break of one minute, followed by a continuous infusion with 0.015 mg/kg (˜1 mg)/min over 84 minutes. The high dose consisted of a bolus injection of 0.3 mg/kg (˜23 mg) and a continuous infusion with 0.02 mg/kg (˜1.5 mg)/min. The two doses were administered on the same day with a two-hour break between the end of the first and the beginning of the second administration in a single-blind design. Subjective effects were assessed and plasma concentrations of DMT were determined during the perfusion and thereafter. Effects of both doses included vivid alterations of visual, auditory, and tactile perception. Visual hallucinations were reported by seven persons already at the low dose and by all participants at the high dose. Most subjects tended to report their experiences during the experiment spontaneously, and they were interested in interpersonal interactions. Mood varied from anxious and tense to expansive and euphoric with mostly vivid verbal, mimic, and psychomotor expression of emotions. Ego-control and insight into the experimental nature of the experience were preserved at the low dose. At the high dose, all subjects reported experiences of altered meaning or significance and developed transient paranoid thoughts and misinterpretations of the experimental situation. Among the 15 subjects, 3 stopped after the first DMT dose due to adverse responses including unpleasant psychological effects (1), nausea (1), and hypotonia (1). These adverse effects vanished within a few minutes from stopping the infusion. No additional medication was given, and no lasting effects were observed. One person stopped due to a headache after both DMT doses. Mean plasma levels of DMT were 43±26 ng/mL and 60±28 ng/mL after the low and high dose, respectively. Plasma levels 10 minutes before the start of the second dose (110 minutes after stopping the first infusion) had dropped to 5±3 ng/mL (Gouzoulis-Mayfrank et al., 2005). This very crude and possibly invalid data would indicate an elimination half-life of approximately 30 minutes. However, the subjective effects subsided more rapidly within 20 minutes and the true half-life is likely shorter (approximately 10 minutes) and more in line with other data (Strassman & Qualls, 1994; Strassman et al., 1996; Strassman et al., 1994). Tiredness and headache in the evening of the experiment was reported in 10 out of 13 subjects who received DMT. One subject reported sleep disturbance in the night after DMT administration. One subject reported mild orthostatic complaints, and another reported two very short episodes of visual perceptual distortions (less than a minute each) in the morning after the DMT experiment. Interviews seven days and 12 months after the experiments revealed no lasting complaints. Overall, the reported effects from this study at the high dose were later evaluated as relatively strong DMT effects, possibly due to a rather high perfusion dose of 1.5 mg/min and lead to a lower suggested rate of 1 mg/min (Gallimore & Strassman, 2016).

Accordingly, the high dose in the present invention can be a 1 mg/minute infusion rate. A 90 minute infusion duration has been selected similar to the one used by (Gouzoulis-Mayfrank et al., 2005). Assuming an elimination half-life of 10 minutes, this administration schedule results in steady-state concentrations after approximately 45 minutes. Steady state is reached after 4-5 elimination half-lives and likely varies individually. The exact half-life values and associated times to reach steady state can be defined in a population of human subjects as part of the present invention generating also reference data for the future use of DMT using the presently described method. The present method allows for reaching specific states rapidly and to maintain and adjust them if needed. In particular the method is suitable to induce stable and controlled psychedelic states lasting minutes to only a few hours. Such rather short states can be preferred by many patients and therapists over long-lasting sessions induced by oral administration of DMT with a MAO inhibitor or of other psychedelics like LSD or psilocybin.

Strassman et al. performed a dose-response study of intravenous DMT (Strassman & Qualls, 1994; Strassman et al., 1994). The study included 11 healthy subjects with previous hallucinogen experience. Participants had used hallucinogens six to hundreds of times. Two subjects had a history of cocaine dependence and all but two had used MDMA five or more times. The study included a non-blind administration of a very low and high dose (0.04 and 0.4 mg/kg) of DMT before the randomized, double-blind administration of 0.05, 0.1, 0.2, and 0.4 mg/kg DMT. DMT was administered as a bolus (infused over 30 seconds and flushed with 5 mL of saline over the next 15 seconds). Treatments were separated by at least 1 week. Subjective, autonomic, and endocrine effects of DMT and DMT plasma concentrations were repeatedly assessed before and 2, 5, 10, 15, 30 and 60 minutes after drug administration. DMT was found to be fully hallucinogenic at the two higher doses of 0.2 and 0.4 mg/kg (˜15 and 30 mg, respectively). Lower doses were not hallucinogenic; emotional and somatic effects predominated. Effects were felt nearly instantaneously, peaked within 2 minutes after injection, and resolved within 20-30 minutes. DMT produced visual hallucinatory phenomena, bodily dissociation, and extreme shifts in mood. Auditory effects were noted in about half the subjects. At the highest dose, subjects were almost uniformly overwhelmed at the intensity and speed of onset of this dose. All subjects described an intense, rapidly developing, and usually transiently anxiety-provoking “rush” throughout the body and mind. Most subjects lost awareness of their bodies, and many were not cognizant of being in the hospital and participating in an experiment for the first minute or two of the experience. Three subjects who had smoked DMT free base agreed that intravenous effects were more overwhelming and rapid in onset at the dose and schedule used in this study (Strassman et al., 1994). Visual imagery predominated in all subjects. Subjects described colors as brighter, more intense, and deeply saturated than those seen in normal awareness or dreams. Subjects initially were anxious as the rush developed. However, they quickly settled into the experience within 15-30 seconds after the injection. The 0.2 mg/kg dose was described as the threshold dose for hallucinogenic effects. The 0.2 mg/kg dose was described as less frightening and resulting in a lower intensity of the experience compared with the 0.4 mg/kg dose. The lower doses were not perceived as pleasant. The time course of DMT blood levels matched the march of subjective effects. Blood levels of DMT base after the highest 0.4 mg/kg dose were ˜90, 42, 28, 17, and 5 ng/mL at 2, 5, 10, 15, and 30 minutes after the DMT bolus administration, respectively. This supports the approach of the present invention to precisely determine the time course of DMT blood levels and subjective effects and use either value also as a surrogate measure of the other to optimize dosing and treatment.

A more recent pilot study also assessed plasma concentrations of DMT after intravenous bolus administration of 7-20 mg of DMT in a small number of subjects (Timmermann et al., 2019). DMT blood concentrations were approximately 56 ng/mL 2 minutes after administration of 14 mg (corresponding to approximately 0.2 mg/kg) (Timmermann et al., 2019) in line with the aforementioned data by Strassman (Strassman et al., 1994). The measurements from both studies are consistent with an approximate elimination half-life of 5-10 minutes. However, this may represent rapid redistribution and metabolism and may therefore be shorter than the true elimination half-life (see above). The study only used a bolus, so it is different from the present invention. The above descriptions illustrate that there is limited information on the effects of intravenous DMT administration. However, no study has validly determined the elimination half-life of DMT and other pharmacokinetic parameters and this will be done within the current invention to have better and rational basis on dosing.

DMT increased blood pressure, heart rate, pupil size, and body core temperature as well as blood levels of ACTH, cortisol, PRL, β-endorphin, and growth hormone. Average heart rate levels and mean arterial blood pressure were 100 beats/min and 108 mm Hg at 2 minutes and declined rapidly. Strassman et al. then performed another study exploring tolerance to DMT and administering DMT at a dose of 0.3 mg/kg intravenously at half-hour intervals 4 times in a morning to 13 experienced hallucinogen-using volunteers (Strassman et al., 1996). The 0.3 mg/kg dose produced blood DMT levels of ˜70, 50, 30, and 18 ng/mL after 2, 5, 10 and 15 minutes with little to no difference between repeated doses. Tolerance to the subjective effects of DMT did not occur. However, the study tested only repeated doses within the same day. Similarly, no acute tolerance to the effect of LSD (Dolder et al., 2015; Holze et al., 2019) were noted while repeated doses at daily intervals were associated with tolerance (Abramson et al., 1956; Cholden et al., 1955; Wolbach et al., 1962). Finally, Strassman et al. investigated the role of the 5-HT1A receptor in the action of DMT. Twelve subjects received a sub-hallucinogenic dose of 0.1 mg/kg DMT in combination with the 5-HT1A receptor blocker pindolol or placebo. Volunteers found that pindolol pre-treatment enhanced DMT effects two to three times in contrast to similar studies in animals (Strassman, 1996). This finding would indicate that activation of 5-HT1A receptors counteracts the psychedelic effects that are primarily mediated via 5-HT2A receptor stimulation. However, this finding and the potential role of the 5-HT1A receptor also needs further confirmation and investigation which are not the focus of the present invention. Based on the experimental studies using DMT intravenously, Gallimore and Strassman proposed a target-controlled intravenous infusion model for a prolonged immersive DMT psychedelic experience (Gallimore & Strassman, 2016). The goal was to design a theoretical infusion protocol that maintains an effect site (brain) concentration of ˜100 ng/mL in a 75 kg subject with the concentration producing a full experience calculated at 60 ng/mL. Based on the data using a 0.4 mg/kg bolus and using a simulated time course of DMT brain concentrations, this dose would result in breakthrough into the DMT space after 1 minute and exit from the DMT space at 8 minutes (Gallimore & Strassman, 2016). To produce a longer effect, the authors proposed to combine a 25 mg (0.3 mg/kg) DMT bolus over 30 seconds that brings the effect site concentration to just over 100 ng/mL with an infusion to maintain the target concentration at ˜100 ng/mL. Although the initial plasma concentration spikes shortly at over 200 ng/mL, the desired effect site concentration is reached smoothly with very little overshoot. Then, to keep the concentration, the authors propose an infusion beginning at 2 minutes at a rate of 4.2 mg/min which would then be reduced every minute to 0.93 mg/min over 20 minutes when steady state concentrations would be reached.

The present invention uses modified administration schemes. The schemes are more practical than proposed previously using only one fixed bolus dose and one fixed continuous perfusion dose as starting point. Additionally, four specific dosing schemes using two different perfusion doses with and without a bolus are specifically tested and put into practice within this invention (Example 1). In contrast, the prior art was essentially a theoretical model that has never been tested and implemented in vivo in humans. Only this data and putting into practice allows one to generate reference data including information on the specific plasma concentrations of DMT and desired states in humans when using specific doses. Only this experimental information derived from a human study can allow for valid dosing recommendations when using this invention.

A method of inducing a psychedelic state in an individual includes a continuous administration of DMT to an individual (through perfusion) and an optional initial bolus administration to the individual. The continuous administration includes the administration of DMT at a dose 0.1-5 mg/min. A typical dose is 0.5-2 mg/min. A bolus can also be used at or before the start of the continuous perfusion of DMT. A bolus dose is 1-100 mg. A typical bolus dose is 5-50 mg. The invention illustrates (FIG. 2) typical dosing examples including a description of the estimated plasma concentrations reached with such dosing.

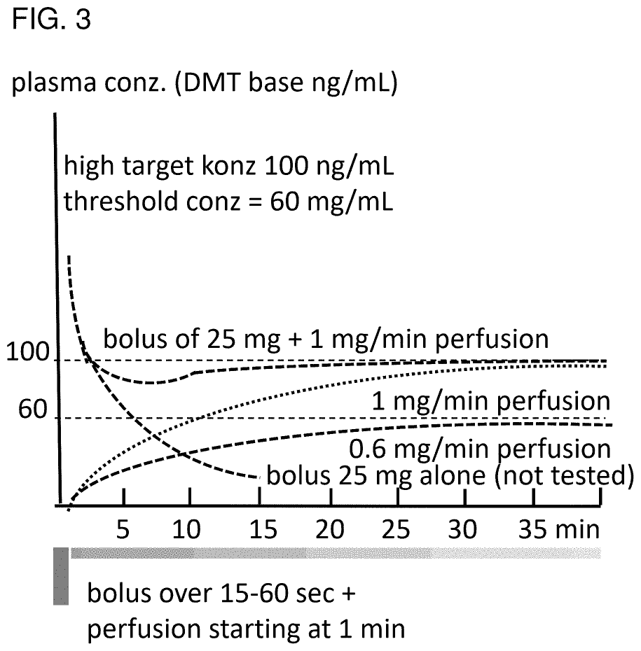

For example, administration of 1 mg/min DMT fumarate can be used and results in an estimated plasma concentration of 100 ng/mL in a human subject and an intense DMT experience. The steady-state (maximal) concentration is reached after about 45 minutes (30-60 minutes). Similarly, it will take 30-60 minutes until the subjective DMT experience will be maximal (FIG. 3). A bolus dose of 25 mg of DMT fumarate can be administered over 30-60 seconds before the continuous perfusion of DMT allowing to reach maximal DMT concentrations faster (FIG. 3). The initial spike in plasma concentrations of DMT does not result in similarly high concentrations in the brain (effect compartment). Thus, the subjective DMT effect establishes faster than with a perfusion only but without an initial peak (unlike the plasma concentration) in FIG. 3. These dosing instructions are examples and other doses for the bolus and perfusion can be used. Also, the duration of the perfusion can be anything between a few minutes to up to several hours (5 minutes-5 hours). A typical duration is 30-90 minutes. In the examples illustrated here and specifically tested a duration of 90 minutes is used and the total mg DMT amounts for such dosing schedules are provided in TABLE 1 as examples.

For the highest dose, the invention specifically tests a bolus using the moderately high 0.3 mg/kg=25 mg bolus proposed by the target-controlled model (Gallimore & Strassman, 2016) and used also by (Strassman et al., 1996). This bolus dose is expected to result in a full DMT experience, but it is lower than the full and overwhelming dose used previously (0.4 mg/kg or 30 mg) (Gouzoulis-Mayfrank et al., 2005; Strassman & Qualls, 1994; Strassman et al., 1994).

The invention uses a bolus infusion time of 45-60 seconds largely similar to the 30 second bolus followed by the 5 mL saline flush over 15 seconds used by Strassman et al. (Strassman et al., 1994). Then after the bolus of 25 mg and starting at minute 1, the perfusion starts with a rate of 1 mg/min (90 mg/90 min) resulting in a total dose of DMT of 115 mg. The state induced by this procedure is stable (in steady state regarding the plasma concentration) after about 45 minutes. The state can be stopped or prolonged as needed. The invention can use 90 minutes duration testing, but this time of perfusion duration and effect duration can be altered. Thus, the invention results in a psychedelic state that is stable after approximately 45 minutes and can be extended as needed for several hours. This is unique to the present invention and an unexpected and critical step forward in the induction and management of psychedelics states similar to the enhanced control over anesthetic and hypnotic states when using total intravenous anesthesia with rapidly acting and controllable compounds during surgery using intravenous perfusion as compared with inducing anesthesia with a pain pill and a sleeping aid. With all previous methods of inducing psychedelic states, typically with oral administration, the state intensity is reached relatively rapidly and then slowly declines following the plasma concentration-time curve of the psychedelic substance and the intensity cannot be influenced once the substance is ingested. Thus, no other administration method using psychedelics orally or as singly doses parenterally is capable of inducing a similar stable state as the present invention. The state can also be stopped rapidly and is then expected to be completely normalized within 45 minutes when all DMT is fully metabolized. In fact, subjective effects of DMT are expected to be almost absent within a shorter time of 10-30 minutes as will be further tested and defined with the human studies underlying this invention.

A low dose of DMT used as an example (FIG. 2) in this invention can be 60% of the high dose tested and include a bolus of 15 mg followed by a perfusion at a rate of 0.6 mg/min over 90 minutes (54 mg) equivalent with a total dose of 69 mg. This low dose bolus corresponds to the dose previously defined as threshold dose for perceptual alteration (psychedelic threshold dose) (Gallimore & Strassman, 2016; Strassman et al., 1994). Both the low and the high dose perfusion can be administered without the bolus (examples in FIG. 2). This allows for a slower induction of the psychedelic state. The advantage of using no bolus as part of the present invention is that the psychedelic state establishes more slowly and with a lower expected risk of anxiety and feeling overwhelming. On the other hand, giving no bolus does not allow for the rapid induction and production of the peak experience and also takes more time. Both approaches are included in the present invention for their specific benefits. For example, no bolus can be used in a person with anxiety or using DMT for the first time to get more slowly experienced with the unfamiliar state of mind. A bolus can be used in a person already experienced in the DMT state and/or in a person where a stronger peak DMT experience is required or desired. This could include situations where greater ego-dissolution is desired such as in a patient suffering from chronic pain. A stronger experience or even near-death experience may be induced with a higher bolus and could be indicated in patients with fear of death to mitigate anxiety. Different situations and disorders require different dosing. This can easily be accomplished with the present invention and the dose can even be titrated to induce specific states during a session.

Further uses of the invention are listed here as examples:

In a person with no experience, a first session treatment can use a low dose of DMT and no bolus. Using this approach mainly positive acute subjective effects are induced. “Positive acute effects” as used herein refers primarily to an increase in subjective rating of “good drug effect” and can also include ratings of “drug liking”, “well-being”, “oceanic boundlessness”, “experience of unity”, “spiritual experience”, “blissful state”, “insightfulness”, any “mystical-type experience” and positively experienced “psychedelic effects”, and “aspects of ego-dissolution” if experienced without anxiety.

Higher doses of DMT can be desired in some persons. In the case of strong negative subjective acute effects, the dose can be adjusted on-the-go by reducing the speed of the perfusion. “Negative acute effects” as used herein refers primarily to subjective ratings of “bad drug effect” and “anxiety” and “fear” and may additionally include increased ratings of “anxious ego-dissolution”, or descriptions of acute paranoia or states of panic and anxiety as observed by others.

The present invention also provides for a method of determining a dose of DMT for an individual, by administering different rates of perfusion of DMT to an individual and adjusting the dose to provide more positive acute effects than negative acute effects in the individual. The individual can be a healthy subject and the method can be used to predict doses for patients. This method can be used to determine long term DMT dosing and dose schedules. For example, a “good drug effect” dose may be selected to be used first followed by a “ego-dissolution” dose later once the subject or patient is used to the effects of DMT. In addition, dose-finding for clinical trials is difficult and time and money consuming. It would be much easier and cost-effective and rapid if a method were available to define the dose of DMT to be used in patients already in Phase 1 studies in healthy subjects. Evaluating the acute effects of DMT in healthy subjects with a focus on positive acute over negative effects as a documented predictor of long-term outcome in patients can greatly facilitate the dose-finding for future Phase 2 and Phase 3 studies in patient populations. Therefore, this method can be used in predicting and determining DMT doses for clinical trials.

The present invention provides for a method of inducing a psychedelic state in an individual safely, by administering DMT, a salt of DMT, analogs thereof, or derivatives thereof to an individual with a continuous perfusion system, inducing a psychedelic state, and adjusting or ending the psychedelic state on demand.

The present invention also provides for a method of providing a short lasting psychedelic treatment of minutes to 1-2 hours, by administering DMT, a salt of DMT, analogs thereof, or derivatives thereof to an individual with a continuous perfusion system and providing psychedelic treatment for minutes to 1-2 hours.

The present invention provides for a method of therapy, by administering an intermediate “good effect dose” of DMT to an individual and inducing positive acute drug effects that are known to be associated with more positive long-term responses in psychiatric patients. The method can be used in treating various medical conditions including depression, anxiety, substance use disorder, other addiction, personality disorder, eating disorder, post-traumatic stress disorder, obsessive compulsive disorder, various pain disorders, migraine, cluster headache, and palliative care.

The present invention also provides for a method of therapy, by administering a higher “ego-dissolution” dose of DMT to an individual and providing ego-dissolution. This method is appropriate for individuals experienced with lower good effect doses of DMT or other psychedelics and aiming for a more intense and ego-dissolving experience but also ready to risk experiencing greater anxiety when dealing with this state. Ego-dissolution as experience may be therapeutic in some indications namely in individuals with severe pain disorders, with cancer and/or in palliative care with the goal of being free of pain or at least not realizing somatic pain and the presence of the body or feeling out of the body during this experience. Ego-dissolution can also be a therapeutic experience in other disorders including personality disorder (narcissistic personality disorder) or as needed by psychiatric indications.

The invention is based on a specific setting consisting of only one or very few patients present during the administration of DMT. The persons or patients treated are not in acute psychological distress prior to the dosing of DMT. The persons do not have an increased risk of psychosis (schizophrenia). The persons are not under the acute influence of other psychoactive substances. The person is comfortably resting in a controlled quiet environment protected from loud noise, other persons (besides from 1-3 supervisors/therapists) with the option of playing suitable music and wearing eye shades or closing eyes.

The doses provided here refer to DMT fumarate salt doses. DMT may be administered in other salt formulations and the dose would need to be adjusted according to the molecular weight to obtain equimolar doses of DMT base and comparable DMT plasma levels. Any other water soluble salt of DMT could be used.

In contrast to the model proposed by Gallimore and Strassman, the present invention does not necessarily involve higher perfusion rates from minutes 2-20 and simply starts the constant perfusion at minute 1. This is expected to result in transiently lower target site concentrations at minutes 2-20 after the bolus dose and until steady state of the constant perfusion administration has developed (greater than 20 minutes). On the other hand, the invention uses a simpler dosing schedule that is considerably more practical.

The key aim of the studies conducted to support the present invention is to define the dose-response of DMT as well as the difference between the loading dose bolus and no-bolus perfusion conditions regarding pharmacokinetic, subjective, and autonomic effects including psychological and physical tolerability. The proposed dosing schedules are similar to the one proposed by Gallimore and Strassman but with more practical perfusion rates. For safety reasons, the perfusion can be stopped at any time in case adverse effects require it.

The use of the present invention includes administering a dose of DMT at a constant rate after a bolus dose or no bolus dose of DMT. The use also includes asking the patient/subject to rate its subjective drug effects on a scale from 0-10 to get a feedback on the dosing. Thus, subjects are asked by the physician or supervisor to repeatedly rate their subjective effects verbally on a Likert scale from 0 to 10 for: “any drug effect”, “good drug effect”, “bad drug effect”, and “fear”. Ratings are performed before and repeatedly after substance administration and will take approximately 30 seconds to be completed. A similar method was used previously to assess DMT effects (Riba et al., 2015) and is less demanding than completing self-rated VAS in written form (Holze et al., 2019) and therefore interferes only minimally with the subjective experience. The ratings are administered repeatedly throughout the study session. The suggested measure within this invention is simple and their performance allows informing the physicians about the state of the patient and allowing feedback and dose adjustments (lowering or increasing the perfusion rate to adjust the psychedelic state based on the verbal feedback).

The possibility of verbal feedback and dose adjustment during the development of a psychedelic state is a key feature of the present invention allowing immediate feedback and on-the go adjustment by the patient and physician.

Additionally, more measures of the psychedelic state can be obtained at the end of the treatment session then allowing to adjust a subsequent treatment session dosing taking place immediately or any day after the first session. Measures proposed for such assessments after the session are for example: 1) the Adjective Mood Rating Scale (AMRS): The adjective mood rating scale (AMRS or EWL60S) is a 60-item Likert scale that allows repeated assessment of mood in 6 dimensions: Activation, inactivation, well-being, anxiety/depressed mood, extra- and introversion, and emotional excitability. The scale is once before and once at the end of a session. The AMRS consists of subscales measuring “activation”, “positive mood”, “extraversion”, “introversion”, “inactivation”, and “emotional excitability.” 2) the 5-Dimensional Altered States of Consciousness (5D-ASC). The 5D-ASC Scale is a questionnaire containing visual analog scales for 94 items (Dittrich, 1998; Studerus et al., 2010). The instrument contains five scales assessing mood, anxiety, derealization, depersonalization, changes in perception, auditory alterations, and reduced vigilance. The scale is well-validated (Studerus et al., 2010) and used internationally to evaluate effects of many other psychoactive substances. The 5D-ASC scale is administered once at the end of the session and subjects will be instructed to retrospectively rate peak alterations that have been experienced during the study session. Each item of the scale is scored on a 0-100 mm VAS. The attribution of the individual items to the subscales of the 5D-ASC is analyzed according to (Dittrich, 1998; Studerus et al., 2010). 3) the States of Consciousness Questionnaire (SCQ). In the SCQ, 100-items are rated on a six-point scale. Forty-three items embedded into this questionnaire comprise the Mystical Experience Questionnaire (MEQ) (Griffiths et al., 2006; MacLean et al., 2011; Pahnke, 1969) which is sensitive to the effects of hallucinogens including LSD (Liechti et al., 2017), psilocybin (MacLean et al., 2011), and DMT (Riba et al., 2015; Timmermann et al., 2018). The 43 items provide scale scores for each of seven domains of mystical experiences: Internal unity (pure awareness, a merging with ultimate reality), external unity (unity of all things, all things are alive, all is one), sense of sacredness (reverence, sacred), noetic quality (encounter with ultimate reality, more real than everyday reality), transcendence of time and space, deeply felt positive mood (joy, peace, love), paradoxicality/ineffability (claim of difficulty in describing the experience in words). The four scale scores derived from the newly validated and revised 30-item MEQ can be used: Mystical, positive mood, transcendence of time and space, and ineffability (Barrett et al., 2015). The MEQ is the main outcome measure for the mystical-type effects as this scale has become a standard measure in hallucinogen research (Barrett et al., 2015; Garcia-Romeu et al., 2015; Griffiths, 2016; Liechti, 2017; Riba et al., 2015). Data on each domain scale is expressed as a percentage of the maximum possible score. Criteria for a “complete” mystical experience are scores on each of the following six scales of at least 60%: External or internal unity, sense of sacredness, noetic quality, transcendence of time, positive mood, and ineffability. 4) the Spiritual Realm Questionnaire (SRQ). The SRQ (K. Stocker, 2020, unpublished) assesses the entheogenic potential of psychedelic substances by connecting the experience of religio-psychological and spiritual phenomena to sensemaking, salubrity, and episteme. The scale covers four constructs (1. phenomenological religio-psychological spirituality spectrum of humanity; 2. human condition and life meaningfulness; 3. dealing with personal problems; 4. worldview/belief) through 11 basic questions in a binary yes/no format and their 65 sub-questions to be answered on a visual analog scale. In addition to the SRQ, a brief 8-item scale (pSRQ) can be used prior to the substance experience to assess the person's inherent worldview (spiritual, materialistic, agnostic). The invention includes the generation or reference effect ranges in all these measures after 4 different DMT treatment regimes as a starting point for adjusted dosing in patients. The invention can also provide reference effect values for autonomic measures of safety (blood pressure and heart rate) and for adverse effects. The list of complaints (LC) consists of 66 items offering a global score measuring physical and general discomfort (Zerssen, 1976). The LC list is ideally used before and at the end of the session with reference to complaints throughout the entire session. Subjects are additionally asked to report any adverse events during the sessions.

Importantly, the measures suggested to assess the psychedelic state have already been used with other psychedelics and have in some case been shown to predict the long-term therapeutic effects of psychedelics. Thus, they serve as an instant marker of the therapeutic longer-term effects of DMT within this invention.

Another key feature of the present invention is establishing and using a link between plasma levels of DMT and subjective effects to have reference values for further dosing improvements and to be used in special cases. Such cases could be patients not responding to DMT in which case a dose increase could be first used. If a response is still minimal, plasma levels of DMT should be determined and compared with the reference values established within the present invention. This allows for distinguishing patients with a small response due to insufficient concentrations (extensive metabolism) from patients with normal concentrations but a tolerance or pharmacodynamically insufficient response. It is expected that higher DMT concentrations at steady state will be associated with greater subjective effect scores. Additionally, substance concentrations measured after the discontinuation of the DMT perfusion allows for the determination of the elimination half-life of DMT. Furthermore, DMT concentrations taken at the beginning of the perfusion phase allows for the characterization of the pharmacokinetics of the bolus-high dose perfusion vs. no-bolus condition. Plasma concentrations of DMT after oral, intravenous, smoked, or intramuscular administration have previously been measured (Kaplan et al., 1974; Riba et al., 2015; Strassman et al., 1994) but the pharmacokinetic parameters have only poorly been characterized (no valid date on half-lives etc.). The present invention can assess the concentrations of DMT repeatedly at close intervals over time and provide a full description of the concentration-time course, pharmacokinetic parameters, and also blood levels of the main DMT metabolites (indole-3-acetic acid (Ormel et al.) and N,N-dimethyltryptamine-N-oxide (DMT-NO)(Riba et al., 2015).

Perceptual changes after administration of DMT include illusions, pseudo-hallucinations, intensification of color perception, metamorphosis-like changes in objects and faces, kaleidoscopic or scenic visual imagery, synesthesia and alterations in thinking and time experience. Body perception is altered involving changes in body image, unusual inner perception of bodily processes and metamorphic alterations of body contours. Given the intermediate DMT dose to be administered in the present invention, subjects are expected to retain their thought control throughout the experience with the exception of the first 2 minutes during the bolus administration and, in contrast to psychotic patients, to remain aware of the transient state of the drug-induced experience. Ego dissolution phenomena are expected to emerge, but only rudimentarily after the administration of a moderate dose as used in the present study. The subjective effects of psychedelics are generally positively evaluated when the experience has taken place in a controlled clinical setting, with healthy subjects and patients displaying similar positive evaluations (Dolder et al., 2016; Gasser et al., 2014; Passie et al., 2008; Schmid et al., 2015). However, side effects like transient dysphoria, anxiety or mood swings might occur (Dolder et al., 2017). In laboratory studies using hallucinogens, mild or moderate anticipatory anxiety is common at the beginning of the onset of a hallucinogenic drug effect (Griffiths et al., 2006). Dysphoria, anxiety and mild, transient ideas of reference/paranoid thinking may also occur in some subjects and can readily be managed with reassurance (Griffiths et al., 2006). Negative experiences (bad trips) and flashback phenomena may occur in uncontrolled conditions (Strassman, 1984). Under controlled and supportive conditions, the psychedelic experience reportedly had lasting positive effects (Carhart-Harris et al., 2016; Gasser et al., 2014; Schmid & Liechti, 2018). For example, administration of a single dose of a psychedelic was considered a personally meaningful experience having long-lasting subjective positive effects up to 12 months (Schmid & Liechti, 2018).

DMT metabolism depends on the activity of drug-metabolizing enzymes including MAO. Genetic alterations in these enzymes may can be determined prior to dosing and use to further define doses of DMT to be used within this invention. Specifically, subjects with low MAO activity may need lower doses than subjects with high MAO activity. On the other hand, the possibility to adjust the DMT dose during the session is a key feature of the present invention allowing also to adjust for such metabolic differences in the regular case where such differences are not known to the subject or treating physician. Thus, compared to other psychedelic treatments, dosing can be optimized constantly and already during the first session allowing to reach the ideal dose faster than with oral dosing where the exposure to the drug cannot typically be altered once the drug is administered.

There are several advantages of the present invention. Characteristics of the present invention of administering DMT via intravenous perfusion as compared with oral dosing of DMT with a MAO inhibitor or as compared with any other orally administered psychedelic are: 1) fast induction of the psychedelic state, 2) possibility of lowering the intensity of the state after drug administration within the first drug administration, 3) possibility of intensifying the subjective drug state after drug administration within the first drug administration, 4) possibility of rapidly stopping drug administration at any time, 5) possibility of immediate feed-back from the subject to adjust the dosing including any options of “patient-controlled psychedelic intensity” measures. All these features are unique to the present invention when compared to an oral administration of psychedelics.

A unique feature of the present invention is the possibility to adjust the intensity of the psychedelic state during the treatment session. Based on the data generated in the example study (EXAMPLE 1) an individual can be started on the highest dose still well-tolerated by the study population tested in EXAMPLE 1 (i.e. 1 mg of DMT/min). Then, the individual is instructed to indicate whether a higher/lower dose is desired once a steady state is reached. Based on the available data from EXAMPLE 1, a new steady state is reached after approximately 20 minutes after starting the perfusion and more rapidly after stopping the perfusion. Thus, an individual can adjust the dose every 20 minutes up or down and repeatedly until reaching its desired level. This is a particularly advantageous feature for the control of the psychedelic state and a key feature of the present invention.

Therefore, the present invention provides for a method of adjusting a psychedelic state in an individual in real time while effects of DMT have already started in the individual, by adjusting a rate of DMT perfusion to increase or decrease the intensity and/or duration of the psychedelic state based on the individual's feedback or a therapist's assessment of the individual's state.

The compounds of the present invention are administered and dosed in accordance with good medical practice, considering the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants and vehicles. The patient being treated is a warm-blooded animal and, in particular, mammals including man. The pharmaceutically acceptable carriers, diluents, adjuvants and vehicles as well as implant carriers generally refer to inert, non-toxic diluents not reacting with the active ingredients of the invention.

The doses can be single doses or multiple doses within a day or over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The invention is further described in detail by reference to the following experimental examples. These examples are provided for the purpose of illustration only and are not intended to be limiting unless otherwise specified. Thus, the invention should in no way be construed as being limited to the following examples, but rather, should be construed to encompass any and all variations which become evident as a result of the teaching provided herein.

### EXAMPLE 1

A clinical study in healthy subjects is conducted to describe the characteristics of the present invention including the pharmacokinetics and effect profiles after different doses of DMT.

Study design and methods: This study uses a double-blind, placebo-controlled, 5-period cross-over design with 4 different dosing schedules of DMT and placebo. On 5 separate days, subjects will be administered DMT in 4 different doses or placebo in randomized, counter-balanced order and separated by at least 1 week. Interventions are administered in double-blind manner thus in pre-prepared vials of the same volume and in perfusion syringes of the same volume (50 ml) containing different doses/concentrations of DMT and administered at the same perfusion rate to allow for double blinding. The study includes a screening visit (2 h), 5 test sessions each lasting 4 h, and one end of study visit (2 h). The outcome measures are subjective effect ratings on Visual Analog Scales, the 5 Dimensions of Altered States of Consciousness (5D-ASC) scale, adjective mood rating scale (AMRS), mystical-type experiences (SCQ), autonomic effects (blood pressure, heart rate), and plasma levels of DMT. Only healthy subjects are included in the study.

Study drugs: DMT was administered in the form of N,N-dimethyltryptamine hem ifumarate (DMT:fumarate 1:0.5). DMT was obtained from research (Burgdorf Switzerland) as analytically pure substance with a HPLC-confirmed purity of 99.9% and identified as the hemifumarate using qNMR. Intravenous solutions of DMT were prepared by Apotheke Dr. Hysek (Biel, Switzerland) according to Good Manufacturing Practice (GMP). A bolus and a perfusion solution dose unit in saline were prepared containing 5 (5 mg/mL) and 18 mg (18 mg/mL) of DMT hemifumarate, respectively. Identical placebo (saline) was prepared. The solutions were tested for content identity and for sterility (pyrogens, microbiology). The bolus administration included 5 bolus vials containing 0, 15 or 25 mg of DMT administered over 45 sec. The perfusion administration started 60 seconds after the start of the bolus and consisted of 5 perfusion vials solved in saline and injected into the perfusion syringe through a microbiological filter (50 mL perfusion syringe) and administered via a perfusion pump at a precise rate of 48 mL/90 min=32 mL/60 min (0.533 mL/min) and containing 0, 54 or 90 mg of DMT in the 50 mL syringe (including 2 mL dead space in the tubing and resulting in the administration of 48 mL/90 min into the body).

Outcome measures: subjective effects are assessed repeatedly during the DMT administration and at the end of the perfusion.

Subjective Effects Rating (Subjective Effect Scale, SES): Participants will be asked by the investigator to repeatedly rate their subjective effects verbally on a Likert scale from 0 to 10 for: “any drug effect”, “good drug effect”, “bad drug effect”, and “fear”. Ratings will be performed before and repeatedly after substance administration and will take approximately 30 sec complete.

The 5-Dimensional Altered States of Consciousness (5D-ASC) Scale is a questionnaire containing visual analog scales for 94 items (Dittrich, 1998; Studerus et al., 2010). The instrument contains five scales assessing mood, anxiety, derealization, depersonalization, changes in perception, auditory alterations, and reduced vigilance. The scale is well-validated (Studerus et al., 2010) and used internationally to evaluate effects of many other psychoactive substances. The 5D-ASC scale will be administered once at the end of the session and subjects will be instructed to retrospectively rate peak alterations that have been experienced during the study session.

Autonomic measures: Blood pressure and heart rate will be recorded at baseline and repeatedly throughout the session. Blood pressure (systolic and diastolic) and heart rate will be measured with an automatic oscillometric device. Emax will be determined for each of the measures and for each study session.

Adverse effects and list of complaints: The list of complaints (LC) consists of 66 items offering a global score measuring physical and general discomfort (Zerssen, 1976). The LC list is administered before and at the end of the session with reference to complaints throughout the entire session.

Substance concentrations: Plasma levels of DMT will be repeatedly measured (Dolder et al., 2015; Dolder et al., 2017). Substance concentrations serve as an important predictor of the subjective effects. It is expected that higher DMT concentrations at steady state will be associated with greater subjective effect scores. Additionally, substance concentrations measured after the discontinuation of the DMT perfusion will allow the determination of the elimination half-life of DMT. Furthermore, DMT concentrations taken at the beginning of the perfusion phase will allow the characterization of the pharmacokinetics of the bolus-high dose perfusion vs. no-bolus condition. Plasma concentrations of DMT after oral, intravenous, smoked, or intramuscular administration have previously been measured (Kaplan et al., 1974; Riba et al., 2015; Strassman et al., 1994) but the pharmacokinetic parameters have only poorly been characterized (no valid date on half-lives etc.). The present study will assess the concentrations of DMT repeatedly at close intervals over time and provide a full description of the concentration-time course, pharmacokinetic parameters, and also blood levels of the main DMT metabolites (indole-3-acetic acid (Ormel et al.) and N,N-dimethyltryptamine-N-oxide (DMT-NO)(Riba et al., 2015). Determination of DMT and its metabolite plasma concentrations will be performed using validated analytical methods.

Results

Because the study described here is ongoing, only the subjective effects over time are described in a sample of five participants and six administrations. Concentration-time profiles of DMT will be available later once the invention is further developed. In any case, the subjective effects produced with DMT are the primary relevant outcome of relevance for the present invention. The further development of the invention will also include testing of additional lower and higher doses of the perfusion than used in the present examples and thus optimize the dosing for different persons and indications. In the analysis of the present results, only the bolus+perfusion schedule was compared with the perfusion alone schedule and placebo without accounting for different doses.

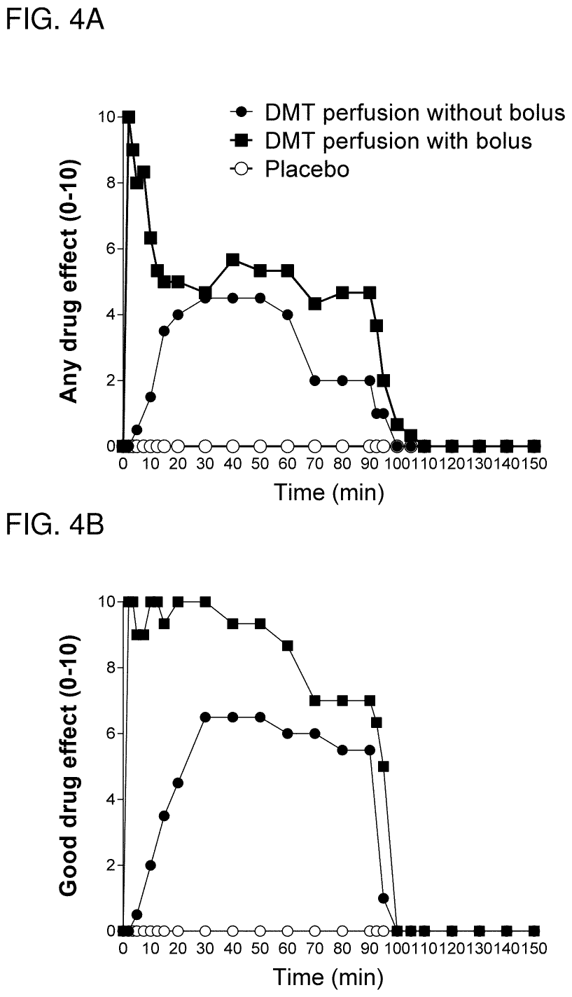

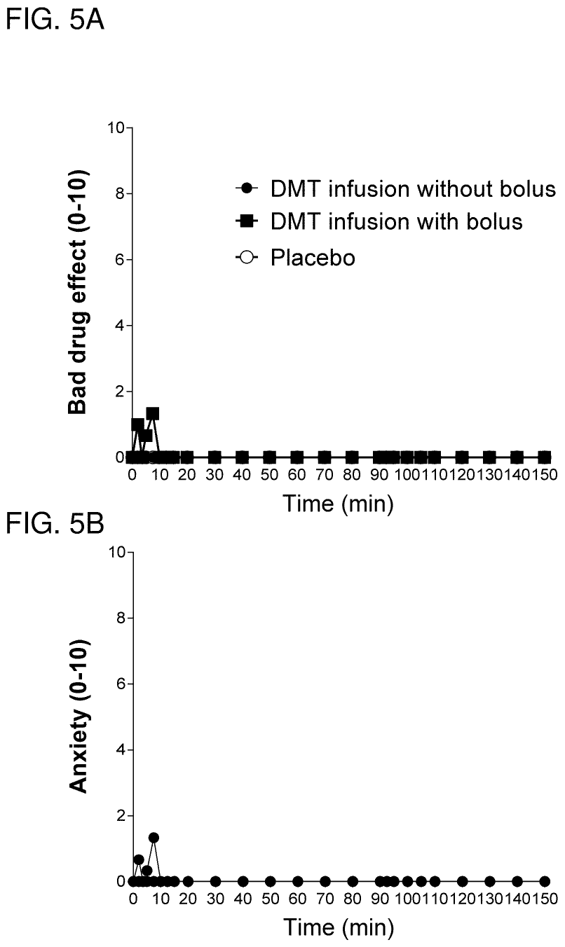

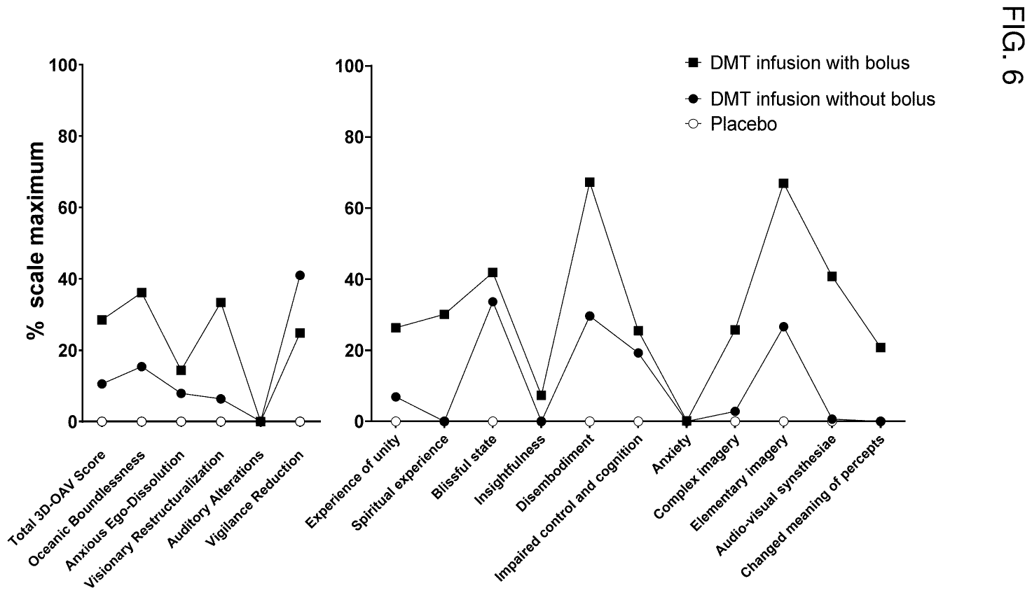

FIGS. 4A-4B show subjective effects of different dosing schedules of DMT. In two research subjects who received DMT as perfusion with a placebo bolus, effects steadily increased over 30 minutes after starting the perfusion and remained elevated (stable in one subject and moderately declining in another) until the perfusion was ended at 90 minutes. Thereafter, the effect rapidly declined and disappeared within less than 10 minutes. Good drug effects steadily increased over 30 minutes after start of the perfusion, staid elevated and relatively stable and rapidly disappeared within less than 10 minutes after stopping the perfusion (FIGS. 4A-4B). There were no bad drug effects and no anxiety at any time during the perfusion of DMT (FIGS. 5A-5B). The DMT experience at the dose used in the example was described as overall similar to LSD at a moderate dose of 0.05-0.1 mg LSD base and characterized by a feeling of detachment from reality and relaxation and feeling carefree without marked visual effects or anxiety or other challenging effects at the dose and dosing schedule used in the example. The doses of the perfusion in the first subjects tested were perceived as rather low by the subjects and investigators and could be increased to obtain the full benefits of the present invention. In three research subjects administered with DMT both as a bolus and perfusion, ratings of “any drug effects” were maximal within seconds of the bolus administration and then declined to reach a plateau during the perfusion (FIG. 4A). The findings are shown as mean in three subjects (FIG. 4A). Ratings of “good drug effects” were also maximal immediately after the DMT bolus administration and up to 30 minutes, then declined slightly during the perfusion and rapidly at the end of the perfusion at 90 minutes (FIG. 4B). There were small increases in ratings of “bad drug effects” and “anxiety” 2-5 minutes after the DMT bolus administration (FIGS. 5A-5B).

The DMT bolus clearly allowed to reach a peak experience very rapidly and lasting for minutes and followed by a plateau phase during the perfusion (FIGS. 4A-4B). If only a perfusion of DMT perfusion was used it took much longer to reach the plateau, but the induction of the experience was smoother and without anxiety and without reaching maximal responses at the doses tested. This administration regime may be preferred in anxious subjects or when using DMT for the first time or if the rapid induction of a full psychedelic state is not desired.

The DMT bolus used in the present first examples was relatively higher compared to the perfusion dose and resulted in maximal responses at the beginning of the session and an overall more altered state (FIG. 6). At the doses tested, the bolus rapidly induced a full derealization and dissociation from realty with intense perceptual alterations. Alternatively, a smaller DMT bolus can be used to still rapidly induce a psychedelic experience compared with the perfusion alone but with a reduced initial peak response compared to the high bolus dose used in EXAMPLE 1. A bolus at the beginning is not mandatory but results in a faster and potentially more intensive acute experience and may be desired in particular in persons and treatment conditions where the aim is to induce “ego-dissolution” and a more comprehensive full psychedelic experience.

FIG. 6 shows alterations of mind induced by different administration schedules of DMT (bolus+perfusion vs. only perfusion) in the 5D-ASC scale. In subjects who received DMT as perfusion with a placebo bolus, DMT produced predominantly positively experienced subjective effects evidenced by increased ratings of Oceanic Boundlessness and lower ratings in Anxious Ego-Dissolution and Visionary Restructuralization. Subscale analysis showed that DMT induced mainly a “blissful state”, “disembodiment” and “impaired control and cognition” and no “anxiety” (FIG. 6). Effects on perception were moderate and included mainly changes in “elementary imaging”. The subject also reported a state of relaxation with no worries and dissociative effects that were experience positively and without anxiety at the dose of DMT used. When DMT was administered as both a bolus and a DMT perfusion it more pronouncedly increased 5D-ASC scores and in all dimensions compared with the perfusion alone. Example results are shown here as mean scores in three subjects (FIG. 6). The experience was overall stronger and in particular ratings of “visionary restructuralization” were clearly higher compared with the DMT perfusion alone. The overall greater dose and more rapid induction with the combined DMT bolus and perfusion administration resulted in a more psychedelic-typical full response with especially greater perceptual changes and also more ego dissolution compared with the perfusion alone with induced a more mellow state (at the moderate doses used). Anxiety was almost absent despite the strong DMT effect seen with the bolus in all subjects. As expected, placebo had no effect on the 5D-ASC (FIG. 6).

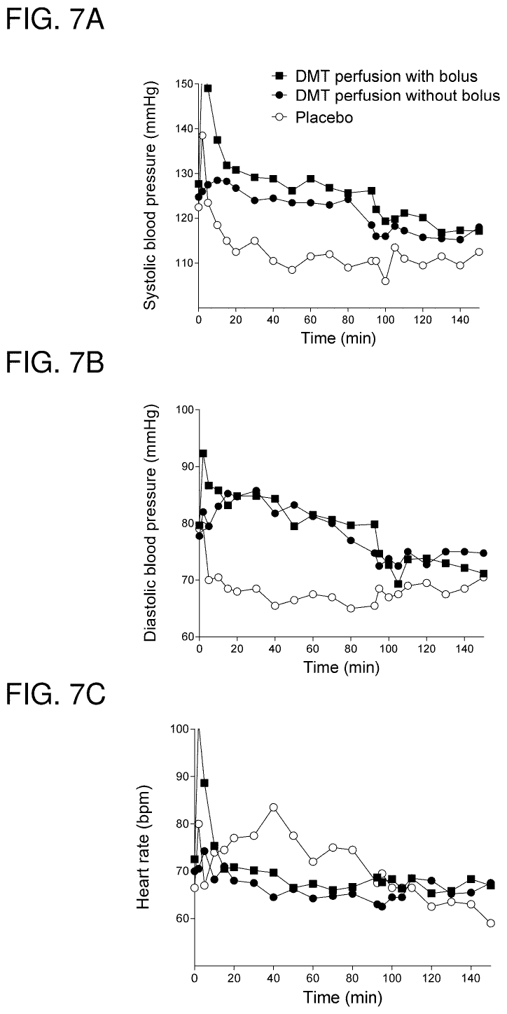

FIGS. 7A-7C show effects of DMT on blood pressure and heart rate. When administered as a perfusion without a bolus or also when administering a bolus in addition to the perfusion, DMT only moderately and transiently increased blood pressure during the time of the bolus administration and perfusion and the bolus produced a transient and slightly greater increase in the cardiovascular response compared to the perfusion only (FIGS. 7A-7C).

Overall, the EXAMPLE illustrates that a psychedelic state can be induced with the present invention and using DMT according to the dosing schedules described and lasting only for a short duration of approximately 90 minutes in the present study or only shortly longer than the perfusion is applied (90 minutes in the present study). Administration of DMT was safe. Administration of DMT did not result in relevant adverse effects. In the example study, two research subjects administered with the perfusion dose of DMT but not the bolus a subject reported a lack of appetite as the only physical change and the other reported tiredness, palpitations, cold feet, and dry mouth during the perfusion and no other complaints. A subject later in the day noted a slight pressure in the head which was not considered a headache or relevant pain. When administered with a bolus and the perfusion, a subject reported more adverse effects including headache, tiredness, lack of appetite, weakness, lack of energy, and difficulty concentrating. These acute effects are comparable to other psychedelics. Another subject administered with the DMT bolus and perfusion did not report any adverse effects. The bolus and/or the total higher dose (bolus plus perfusion) compared with perfusion alone likely increased acute complaints, but these were still moderate and as typically reported also for other psychedelics.

In the example studies presented here (FIGS. 4A-7C) data was included from only a few subjects as illustrations and without differentiating the different doses of the perfusion and bolus used. More date will become available once the present invention is further developed and once the associated studies are completed and fully analyzed.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

1. Abramson H A, Jarvik M E, Gorin M H, & Hirsch M W (1956). Lysergic acid diethylamide (LSD-25): XVII. Tolerance development and its relationship to a theory of psychosis. J Psychol 41: 81-105.
2. Alamia A, Timmermann C, Nutt D J, VanRullen R, & Carhart-Harris R L (2020). DMT alters cortical travelling waves. Elife 9.
3. Barrett F S, Johnson M W, & Griffiths R R (2015). Validation of the revised Mystical Experience Questionnaire in experimental sessions with psilocybin. J Psychopharmacol 29: 1182-1190.
4. Bogenschutz M P, Forcehimes A A, Pommy J A, Wilcox C E, Barbosa P C, & Strassman R J (2015). Psilocybin-assisted treatment for alcohol dependence: a proof-of-concept study. J Psychopharmacol 29: 289-299.
5. Boszormenyi Z, & Szara S (1958). Dimethyltryptamine experiments with psychotics. J Ment Sci 104: 445-453.
6. Carhart-Harris R L, Kaelen M, Bolstridge M, Williams T M, Williams L T, Underwood R, Feilding A, & Nutt D J (2016). The paradoxical psychological effects of lysergic acid diethylamide (LSD). Psychol Med 46: 1379-1390.
7. Cholden L S, Kurland A, & Savage C (1955). Clinical reactions and tolerance to LSD in chronic schizophrenia. J Nerv Ment Dis 122: 211-221.
8. Cozzi N V, Gopalakrishnan A, Anderson L L, Feih J T, Shulgin A T, Daley P F, & Ruoho A E (2009). Dimethyltryptamine and other hallucinogenic tryptamines exhibit substrate behavior at the serotonin uptake transporter and the vesicle monoamine transporter. J Neural Transm 116: 1591-1599.
9. Davis A K, Barrett F S, May D G, Cosimano M P, Sepeda N D, Johnson M W, Finan P H, & Griffiths R R (2021). Effects of psilocybin-assisted therapy on major depressive disorder: a randomized clinical trial. JAMA Psychiatry 78: 481-489.
10. de Araujo D B Acute, lasting and antidepressant effects of Ayahuasca.
11. Dittrich A (1998). The standardized psychometric assessment of altered states of consciousness (ASCs) in humans. Pharmacopsychiatry 31 (Suppl 2): 80-84.
12. Dolder P C, Schmid Y, Haschke M, Rentsch K M, & Liechti M E (2015). Pharmacokinetics and concentration-effect relationship of oral LSD in humans. Int J Neuropsychopharmacol 19: pyv072.
13. Dolder P C, Schmid Y, Mueller F, Borgwardt S, & Liechti M E (2016). LSD acutely impairs fear recognition and enhances emotional empathy and sociality. Neuropsychopharmacology 41: 2638-2646.
14. Dolder P C, Schmid Y, Steuer A E, Kraemer T, Rentsch K M, Hammann F, & Liechti ME (2017). Pharmacokinetics and pharmacodynamics of lysergic acid diethylamide in healthy subjects. Clin Pharmacokinetics 56: 1219-1230.
15. Dominguez-Clave E, Soler J, Elices M, Pascual J C, Alvarez E, de la Fuente Revenga M, Friedlander P, Feilding A, & Riba J (2016). Ayahuasca: Pharmacology, neuroscience and therapeutic potential. Brain Res Bull 126: 89-101.
16. Dong C, Ly C, Dunlap L E, Vargas M V, Sun J, Hwang I W, Azinfar A, Oh W C, Wetsel W C, Olson D E, & Tian L (2021). Psychedelic-inspired drug discovery using an engineered biosensor. Cell 184: 2779-2792.e2718.
17. Dos Santos R G, Balthazar F M, Bouso J C, & Hallak J E (2016a). The current state of research on ayahuasca: A systematic review of human studies assessing psychiatric symptoms, neuropsychological functioning, and neuroimaging. J Psychopharmacol 30: 1230-1247.
18. Dos Santos R G, Osorio F L, Crippa J A, & Hallak J E (2016b). Antidepressive and anxiolytic effects of ayahuasca: a systematic literature review of animal and human studies. Braz J Psychiatry 38: 65-72.
19. Dos Santos R G, Osorio F L, Crippa J A, Riba J, Zuardi A W, & Hallak J E (2016c). Antidepressive, anxiolytic, and antiaddictive effects of ayahuasca, psilocybin and lysergic acid diethylamide (LSD): a systematic review of clinical trials published in the last 25 years. Ther Adv Psychopharmacol 6: 193-213.
20. Faillace L A, Vourlekis A, & Szara S (1967). Clinical evaluation of some hallucinogenic tryptamine derivatives. J Nerv Ment Dis 145: 306-313.
21. Gallimore A R, & Strassman R J (2016). A model for the application of target-controlled intravenous infusion for prolonged immersive DMT psychedelic experience. Front Pharmacol doi: 10.3389/fphar.2016.00211.
22. Garcia-Romeu A, Griffiths R R, & Johnson M W (2015). Psilocybin-occasioned mystical experiences in the treatment of tobacco addiction. Curr Drug Abuse Rev 7: 157-164.
23. Gasser P, Holstein D, Michel Y, Doblin R, Yazar-Klosinski B, Passie T, & Brenneisen R (2014). Safety and efficacy of lysergic acid diethylamide-assisted psychotherapy for anxiety associated with life-threatening diseases. J Nerv Ment Dis 202: 513-520.
24. Gasser P, Kirchner K, & Passie T (2015). LSD-assisted psychotherapy for anxiety associated with a life-threatening disease: a qualitative study of acute and sustained subjective effects. J Psychopharmacol 29: 57-68.
25. Gouzoulis-Mayfrank E, Heekeren K, Neukirch A, Stoll M, Stock C, Obradovic M, & Kovar K A (2005). Psychological effects of (S)-ketamine and N,N-dimethyltryptamine (DMT): a double-blind, cross-over study in healthy volunteers. Pharmacopsychiatry 38: 301-311.
26. Griffiths R (6). Overview of the Johns Hopkins psilocybin research project. In Interdisciplinary Conference on Psychedelics Research. Amsterdam, Jun. 3-5, 2016.
27. Griffiths R R, Johnson M W, Carducci M A, Umbricht A, Richards W A, Richards B D, Cosimano M P, & Klinedinst M A (2016). Psilocybin produces substantial and sustained decreases in depression and anxiety in patients with life-threatening cancer: a randomized double-blind trial. J Psychopharmacol 30: 1181-1197.
28. Griffiths R R, Richards W A, McCann U, & Jesse R (2006). Psilocybin can occasion mystical-type experiences having substantial and sustained personal meaning and spiritual significance. Psychopharmacology (Berl) 187: 268-283; discussion 284-292.
29. Heekeren K, Neukirch A, Daumann J, Stoll M, Obradovic M, Kovar K A, Geyer M A, & Gouzoulis-Mayfrank E (2007). Prepulse inhibition of the startle reflex and its attentional modulation in the human S-ketamine and N,N-dimethyltryptamine (DMT) models of psychosis. J Psychopharmacol 21: 312-320.
30. Holze F, Duthaler U, Vizeli P, Muller F, Borgwardt S, & Liechti M E (2019). Pharmacokinetics and subjective effects of a novel oral LSD formulation in healthy subjects. Br J Clin Pharmacol 85: 1474-1483.
31. Holze F, Vizeli P, Ley L, Muller F, Dolder P, Stocker M, Duthaler U, Varghese N, Eckert A, Borgwardt S, & Liechti M E (2021). Acute dose-dependent effects of lysergic acid diethylamide in a double-blind placebo-controlled study in healthy subjects. Neuropsychopharmacology 46: 537-544.
32. Johnson M W, Garcia-Romeu A, Cosimano M P, & Griffiths R R (2014). Pilot study of the 5-HT2AR agonist psilocybin in the treatment of tobacco addiction. J Psychopharmacol 28: 983-992.
33. Kaplan J, Mandel L R, Stillman R, Walker R W, VandenHeuvel W J, Gillin J C, & Wyatt R J (1974). Blood and urine levels of N,N-dimethyltryptamine following administration of psychoactive dosages to human subjects. Psychopharmacologia 38: 239-245.
34. Kraehenmann R, Pokorny D, Vollenweider L, Preller K H, Pokorny T, Seifritz E, & Vollenweider F X (2017). Dreamlike effects of LSD on waking imagery in humans depend on serotonin 2A receptor activation. Psychopharmacology (Berl) 234: 2031-2046.
35. Krebs T S, & Johansen P O (2012). Lysergic acid diethylamide (LSD) for alcoholism: meta-analysis of randomized controlled trials. J Psychopharmacol 26: 994-1002.
36. Liechti M E (2017). Modern clinical research on LSD. Neuropsychopharmacology 42: 2114-2127.
37. Liechti M E, Dolder P C, & Schmid Y (2017). Alterations in conciousness and mystical-type experiences after acute LSD in humans. Psychopharmacology 234: 1499-1510.
38. Ly C, Greb A C, Cameron L P, Wong J M, Barragan E V, Wilson P C, Burbach K F, Soltanzadeh Zarandi S, Sood A, Paddy M R, Duim W C, Dennis M Y, McAllister A K, Ori-McKenney K M, Gray J A, & Olson D E (2018). Psychedelics promote structural and functional neural plasticity. Cell Rep 23: 3170-3182.
39. MacLean K A, Johnson M W, & Griffiths R R (2011). Mystical experiences occasioned by the hallucinogen psilocybin lead to increases in the personality domain of openness. J Psychopharmacol 25: 1453-1461.
40. Ormel J, Bastiaansen A, Riese H, Bos E H, Servaas M, Ellenbogen M, Rosmalen J G, & Aleman A (2013). The biological and psychological basis of neuroticism: current status and future directions. Neurosci Biobehav Rev 37: 59-72.
41. Pahnke WN (1969). Psychedelic drugs and mystical experience. Int Psychiatry Clin 5: 149-162.
42. Palhano-Fontes F, Barreto D, Onias H, Andrade K C, Novaes M M, Pessoa J A, Mota-Rolim S A, Osorio F L, Sanches R, Dos Santos R G, Tofoli L F, de Oliveira Silveira G, Yonamine M, Riba J, Santos F R, Silva-Junior A A, Alchieri J C, Galvao-Coelho N L, Lobao-Soares B, Hallak J E C, Arcoverde E, Maia-de-Oliveira J P, & Araujo D B (2019). Rapid antidepressant effects of the psychedelic ayahuasca in treatment-resistant depression: a randomized placebo-controlled trial. Psychol Med 49: 655-663.
43. Passie T, Halpern J H, Stichtenoth D O, Emrich H M, & Hintzen A (2008). The pharmacology of lysergic acid diethylamide: a review. CNS Neurosci Ther 14: 295-314.
44. Preller K H, Herdener M, Pokorny T, Planzer A, Kraehenmann R, Staemfli P, Liechti M E, Seifritz E, & Vollenweider F X The role of the serotonin 2A receptor in the fabric and modulation of personal meaning in lysergic acid diethylamide (LSD)-induced states.
45. Riba J, Mcllhenny E H, Bouso J C, & Barker S A (2015). Metabolism and urinary disposition of N,N-dimethyltryptamine after oral and smoked administration: a comparative study. Drug Test Anal 7: 401-406.
46. Rickli A, Moning O D, Hoener M C, & Liechti M E (2016). Receptor interaction profiles of novel psychoactive tryptamines compared with classic hallucinogens. Eur Neuropsychopharmacol 26: 1327-1337.
47. Ross S, Bossis A, Guss J, Agin-Liebes G, Malone T, Cohen B, Mennenga S E, Belser A, Kalliontzi K, Babb J, Su Z, Corby P, & Schmidt B L (2016). Rapid and sustained symptom reduction following psilocybin treatment for anxiety and depression in patients with life-threatening cancer: a randomized controlled trial. J Psychopharmacol 30: 1165-1180.
48. Sanches R F, de Lima Osorio F, Dos Santos R G, Macedo L R, Maia-de-Oliveira J P, Wichert-Ana L, de Araujo D B, Riba J, Crippa J A, & Hallak J E (2016). Antidepressant Effects of a Single Dose of Ayahuasca in Patients With Recurrent Depression: A SPECT Study. J Clin Psychopharmacol 36: 77-81.
49. Schartner M M, & Timmermann C (2020). Neural network models for DMT-induced visual hallucinations. Neurosci Conscious 2020: niaa024.
50. Schmid Y, Enzler F, Gasser P, Grouzmann E, PreIler K H, Vollenweider F X, Brenneisen R, Mueller F, Borgwardt S, & Liechti M E (2015). Acute effects of lysergic acid diethylamide in healthy subjects. Biol Psychiatry 78: 544-553.
51. Schmid Y, & Liechti M E (2018). Long-lasting subjective effects of LSD in normal subjects. Psychopharmacology (Berl) 235: 535-545.
52. Strassman R J (1984). Adverse reactions to psychodelic drugs: a review of the literature J Nerv Ment Dis 172: 577-595.
53. Strassman R J (1996). Human psychopharmacology of N,N-dimethyltryptamine. Behav Brain Res 73: 121-124.
54. Strassman R J, & Qualls C R (1994). Dose-response study of N,N-dimethyltryptamine in humans: I. Neuroendocrine, autonomic, and cardiovascular effects. Arch Gen Psychiatry 51: 85-97.
55. Strassman R J, Qualls C R, & Berg L M (1996). Differential tolerance to biological and subjective effects of four closely spaced doses of N,N-dimethyltryptamine in humans. Biol Psychiatry 39: 784-795.
56. Strassman R J, Qualls C R, Uhlenhuth E H, & Kellner R (1994). Dose-response study of N,N-dimethyltryptamine in humans: II. Subjective effects and preliminary results of a new rating scale. Arch Gen Psychiatry 51: 98-108.
57. Studerus E, Gamma A, & Vollenweider FX (2010). Psychometric evaluation of the altered states of consciousness rating scale (OAV). PLoS One 5: e12412.
58. Szara S (1957). The comparison of the psychotic effect of tryptamine derivatives with the effects of mescaline and LSD-25 in selfexperiments. In Psychotropic Drugs. eds Garattini S., & V. G. Elsevier: Amsterdam.
59. Szara S (2007). DMT at fifty. Neuropsychopharmacol Hung 9: 201-205.
60. Szara S, Rockland L H, Rosenthal D, & Handlon J H (1966). Psychological effects and metabolism of N,N-diethyltryptamine in man. Arch Gen Psychiatry 15: 320-329.
61. Timmermann C, Roseman L, Schartner M, Milliere R, Williams LTJ, Erritzoe D, Muthukumaraswamy S, Ashton M, Bendrioua A, Kaur O, Turton S, Nour M M, Day C M, Leech R, Nutt D J, & Carhart-Harris R L (2019). Neural correlates of the DMT experience assessed with multivariate EEG. Sci Rep 9: 16324.
62. Timmermann C, Roseman L, Williams L, Erritzoe D, Martial C, Cassol H, Laureys S, Nutt D, & Carhart-Harris R (2018). DMT Models the Near-Death Experience. Front Psychol 9: 1424.
63. Vollenweider F X, Vollenweider-Scherpenhuyzen M F, Babler A, Vogel H, & Hell D (1998). Psilocybin induces schizophrenia-like psychosis in humans via a serotonin-2 agonist action. Neuroreport 9: 3897-3902.
64. Winstock A R, Kaar S, & Borschmann R (2014). Dimethyltryptamine (DMT): prevalence, user characteristics and abuse liability in a large global sample. J Psychopharmacol 28: 49-54.
65. Wolbach A, Isbell H, & Miner E (1962). Cross tolerance between mescaline and LSD-25 with a comparison of the mescaline and LSD reactions. Psychopharmacology 3: 1-14.
66. Zerssen D V (1976) Die Beschwerden-Liste. Müchener Informations system. Psychis: München.

What is claimed is:

1. A method of inducing a psychedelic state in an individual, including the steps of:
administering a composition chosen from the group consisting of DMT, a salt of DMT, analogs thereof, and derivatives thereof to an individual with a continuous perfusion system; and
inducing a psychedelic state.

2. The method of claim 1, wherein the composition is administered continuously in a dose of 0.1-5 mg/minute.

3. The method of claim 2, wherein the composition is administered continuously for 5 minutes to 5 hours.

4. The method of claim 3, wherein said inducing step is further defined as inducing a stable and controlled psychedelic state lasting minutes to hours.

5. The method of claim 1, wherein said administering step is further defined as administering the composition to an individual with anxiety or an individual who is using the composition for a first time.

6. The method of claim 1, further including the step of inducing positive acute subjective effects chosen from the group consisting of “good drug effect”, “drug liking”, “well-being”, “oceanic boundlessness”, “experience of unity”, “spiritual experience”, “blissful state”, “insightfulness”, “mystical-type experience”, positively experienced “psychedelic effects”, “aspects of ego-dissolution” if experienced without anxiety, and combinations thereof.

7. The method of claim 1, further including before said administering step, the step of administering a bolus dose of a composition chosen from the group consisting of DMT, a salt of DMT, analogs thereof, and derivatives thereof to the individual.

8. The method of claim 7, wherein the bolus dose of the composition is 1-100 mg.

9. The method of claim 7, wherein the bolus dose is administered over 30-60 seconds.

10. The method of claim 7, wherein the individual has used the composition before or requires a strong peak experience.

11. The method of claim 1, wherein said inducing step is performed rapidly within seconds to minutes.

12. The method of claim 1, wherein said inducing step results in a stable psychedelic state after 45 minutes.

13. The method of claim 1, further including the step of stopping the psychedelic state rapidly within 10-30 minutes.

14. The method of claim 1, further including the step of obtaining rapid/instant verbal feedback from the individual on the intensity of a psychedelic experience.

15. The method of claim 14, further including the step of rapidly adjusting the psychedelic state based on the verbal feedback or other feedback and providing a lower or higher intensity experience within minutes.

16. The method of claim 1, wherein the individual is resting in a controlled quiet environment protected from loud noise and in the presence of a therapist.

17. The method of claim 1, further including the steps of measuring the psychedelic state of the individual at the end of the treatment session and adjusting a dose based on the measurements in a subsequent treatment session.

18. The method of claim 17, wherein said measuring step is further defined as a measure chosen from the group consisting of the Adjective Mood Rating Scale (AMRS), the 5-Dimensional Altered States of Consciousness (5D-ASC), the States of Consciousness Questionnaire (SCQ), and the Spiritual Realm Questionnaire (SRQ).

19. The method of claim 1, further including the step of measuring plasma levels of the composition, and adjusting the dose of the composition in a future treatment session based on the measurement.

20. A method of inducing a psychedelic state in an individual safely, including the steps of:
administering a composition chosen from the group consisting of DMT, a salt of DMT, analogs thereof, and derivatives thereof to an individual with a continuous perfusion system;
inducing a psychedelic state; and
adjusting or ending the psychedelic state on demand.

21. The method of claim 20, wherein said inducing step and said adjusting or ending step can be controlled based on the duration of the continuous perfusion system to last from minutes to a few hours.

22. The method of claim 20, wherein the composition is administered in a dose of 0.1-5 mg/minute for 5 minutes to 5 hours.

23. The method of claim 20, further including before said administering step, the step of administering a bolus dose of 1-100 mg of a composition chosen from the group consisting of DMT, a salt of DMT, analogs thereof, and derivatives thereof to the individual.

24. A method of providing a short lasting psychedelic treatment of minutes to 1-2 hours, including the steps of:
administering a composition chosen from the group consisting of DMT, a salt of DMT, analogs thereof, and derivatives thereof to an individual with a continuous perfusion system; and
providing psychedelic treatment for minutes to 1-2 hours.

25. The method of claim 24, wherein the composition is administered in a dose of 0.1-5 mg/minute for 5 minutes to 5 hours.

26. The method of claim 24, further including before said administering step, the step of administering a bolus dose of 1-100 mg of a composition chosen from the group consisting of DMT, a salt of DMT, analogs thereof, and derivatives thereof to the individual.

27. A method of determining a dose of DMT for an individual, including the steps of:
administering different rates of perfusion of a composition chosen from the group consisting of DMT, a salt of DMT, analogs thereof, and derivatives thereof to an individual; and
adjusting the dose to provide more positive acute effects than negative acute effects in the individual.

28. The method of claim 27, wherein the positive acute effects are chosen from the group consisting of “good drug effect”, “drug liking”, “well-being”, “oceanic boundlessness”, “experience of unity”, “spiritual experience”, “blissful state”, “insightfulness”, “mystical-type experience”, positively experienced “psychedelic effects”, “aspects of ego-dissolution” if experienced without anxiety, and combinations thereof, and wherein the negative acute effects are chosen from the group consisting of “bad drug effect”, “anxiety”, “fear”, increased ratings of “anxious ego-dissolution”, descriptions of acute paranoia, states of panic, and combinations thereof.

29. A method of therapy, including the steps of:
administering an intermediate “good effect dose” of a composition chosen from the group consisting of DMT, a salt of DMT, analogs thereof, and derivatives thereof to an individual; and
inducing positive acute drug effects that are known to be associated with more positive long-term responses in psychiatric patients.

30. The method of claim 29, wherein the positive acute effects are chosen from the group consisting of “good drug effect”, “drug liking”, “well-being”, “oceanic boundlessness”, “experience of unity”, “spiritual experience”, “blissful state”, “insightfulness”, “mystical-type experience”, positively experienced “psychedelic effects”, “aspects of ego-dissolution” if experienced without anxiety, and combinations thereof.

31. The method of claim 29, further including the step of treating a medical condition chosen from the group consisting of depression, anxiety, substance use disorder, other addiction, personality disorder, eating disorder, post-traumatic stress disorder, obsessive compulsive disorder, various pain disorders, migraine, cluster headache, and palliative care.

32. A method of therapy, including the steps of:
administering an “ego-dissolution” dose of a composition chosen from the group consisting of DMT, a salt of DMT, analogs thereof, and derivatives thereof to an individual; and
providing ego-dissolution.

33. The method of claim 32, wherein the individual has a condition chosen from the group consisting of severe pain disorders, cancer, in palliative care, personality disorder, and combinations thereof.

34. A method of adjusting a psychedelic state in an individual in real time while effects of DMT have already started in the individual, including the steps of:
adjusting a psychedelic state in an individual in real time while effects of DMT, a salt of DMT, analogs thereof, and derivatives thereof have already started in the individual, by adjusting a rate of the DMT perfusion to increase or decrease the intensity and/or duration of the psychedelic state based on the individual's feedback or a therapist's assessment of the individual's state.

35. The method of claim 34, further including, before said adjusting step, the step of starting a perfusion with 1 mg of DMT/minute over 10-30 minutes in the individual, and wherein said adjusting step is further defined as the individual deciding whether to increase or decrease the dose to 1.2 mg or 0.8 mg/minute DMT, respectively, and the individual repeating said deciding step again every 10-30 minutes to further optimize the dose to the individual's need.

36. The method of claim 35, wherein said deciding step based on subjective effect strength (any drug effect) and positive versus negative drug effects.
